(19) United States
(12) Patent Application Publication
(10) Pub. No.: US 2019/0099228 A1
(43) Pub. Date: Apr. 4, 2019

(54) Medical Manipulator And Method For Controlling A Medical Manipulator

(71) Applicant: KUKA Deutschland GmbH, Augsburg (DE)

(72) Inventors: Henrik Keller, Augsburg (DE); Tobias Reichl, München (DE); Oliver Thilmann, Augsburg (DE)

(21) Appl. No.: 16/086,558

(22) PCT Filed: Mar. 29, 2017
(86) PCT No.: PCT/EP2017/000379

§ 371 Date: Sep. 19, 2018

(30) Foreign Application Priority Data
Mar. 29, 2016 (DE) 102016205085.0

(51) Int. Cl. A61B 34/30; B25J 15/00; B25J 9/16; A61B 34/00

(52) U.S. Cl. CPC A61B 34/30; B25J 15/0066; G05B 2219/39176; A61B 34/70; B25J 9/1638

(57) ABSTRACT

A medical manipulator includes a manipulator arm, an end effector secured to the manipulator arm and having at least one tool with a changing mass and/or mass distribution, and a manipulator controller for controlling the medical manipulator. In each control step, the manipulator controller uses a current load data matrix including the changing mass and/or mass distribution of the tool to prevent deviations from a target position or target path of the tool.

### CROSS-REFERENCE TO RELATED APPLICATIONS

This application is a national phase application under 35 U.S.C. § 371 of International Patent Application No. PCT/EP2017/000379, filed Mar. 29, 2017 (pending), which claims the benefit of German Patent Application No. DE 10 2016 205 085.0, filed Mar. 29, 2016, the disclosures of which are incorporated by reference herein in their entirety.

### TECHNICAL FIELD

The present invention relates to a medical manipulator and a method for the controlling of a medical manipulator. The medical manipulator can be used, for example, in robotic surgery.

### BACKGROUND

A medical manipulator is used, for example, in robotic surgery for minimally invasive surgical procedures. For these minor procedures, the organism is less severely burdened and any remaining scars are only small in comparison to conventional operation techniques. With the assistance of the medical manipulator, procedures can be performed in and on the human body with a very high level of precision and in tight spaces. A multiplicity of tools, such as a camera system, a scalpel, and a gripper, can be mounted on the arm of a medical manipulator. These tools can be moved in and out of the human body, for example using linear axes through a trocar.

Due to the movement of the tools, however, their mass distribution changes with respect to the manipulator, which may result in an undesired deviation from the target posture or target path of the tool.

In machine-controlled systems, such a deviation is compensated through the minimization of the contouring error of powered mechanical elements, for example the aforementioned linear axes. However, here, the deviation must first appear before the manipulator control can react to it. The appearance of such deviations already impairs the precision of the manipulator and is therefore undesirable.

For hand-operated manipulators, the change in the mass distribution of the tool is interpreted by the manipulator control as a force emanating from the hand of the user. Accordingly, a user must permanently, manually counteract this force. In the case of long hand-operated work phases, in particular, this is ergonomically disadvantageous and leads to a tiring of the user.

It is therefore the object of the present invention to provide a medical manipulator and a method for the controlling of a medical manipulator that overcomes the aforementioned problems. In particular, the deviations of the tool from the target posture or target path should be reduced in order to increase the precision of the manipulator and provide an ergonomic usage in the case of hand-operated manipulator control.

### SUMMARY

The aforementioned object is inventively solved by a medical manipulator and a method for the controlling of a medical manipulator as shown and described herein.

The aforementioned object is solved, in particular, by a medical manipulator comprising a manipulator arm, an end effector mounted to the manipulator arm and comprising at least one tool with a varying mass and/or mass distribution, and a manipulator control for controlling the medical manipulator, wherein the manipulator control uses a current load data matrix with the varying mass and/or mass distribution in each regulation step in order to prevent deviations from the target posture or target path of the tool. The mass distribution of the tool can vary in that the tool is moved with respect to the manipulator. As described above, this can occur by way of insertion and extraction of tools into and out of the human body. The mass of the tool can vary if, for example, objects are picked up with a gripper or, for example, in that objects or materials are inserted into the body and deposited there, such as stents in vascular surgery or staples for the closing of wounds. The mass of the tool can vary in that saltwater is pumped into a balloon in order to expand it. Because the manipulator control uses a current load data matrix in each regulation step, the current values of the mass and mass distribution of the tool are always taken into account in the regulation, whereby a deviation from the target posture or target path of the tool is prevented. Additionally, the posture of the manipulator is precise, because the current load data matrix is updated in each regulation step.

The manipulator control preferably uses a dynamic model of the tool, the calculation of which includes the varying mass and/or mass distribution of the tool. Changes in the mass and/or mass distribution appearing as a result of the movement of the mechanical elements are taken into account in the dynamic model, and the manipulator control can be adjusted accordingly. With the assistance of the dynamic model, the load data can also be determined with a higher temporal resolution in the manipulator control. Additionally, the manipulator control can determine and take into account future values from the dynamic model, in particular the values of the mass distribution of the tool.

The dynamic model of the tool is preferably integrated in the dynamic model of the manipulator. All movements of the manipulator are thereby described in a common model, which prevents communication errors and improves the overall control of the manipulator.

The manipulator preferably takes into account the knowledge regarding a change of the mass and/or mass distribution of the tool. Preparatory calculations can thereby be performed, and constant values can be detected. This facilitates subsequent calculations and makes the data processing faster overall. Additionally, a deviation that occurs during a time step (regulator cycle) is corrected and thus reduced, and not only after the fact.

The end effector preferably comprises a multiplicity of tools, which can be moved independently of one another with respect to the manipulator arm. Through the simultaneous deployment of multiple tools, such as a camera and a gripper, the user of the manipulator is aided in that he or she gains, for example, better views of the operation area. Additionally, more complex tasks can be performed, in that, for example, a gripper holds an opening free and a scalpel is used in the opening simultaneously. Due to the independent movement of the tools, is it possible to selectively insert only certain tools into the respective trocar, which increases visibility during the work.

The mass distribution is preferably calculated through the known position of one or more mechanical elements of the tool and then fed to the manipulator control.

The calculation of the mass distribution in the manipulator control can also occur directly in the tool (end effector). The mass distribution can be calculated indirectly in advance, so that deviations from a target posture or target path of the tool are prevented from the outset.

An electronic control unit that controls the mechanical elements preferably transmits a notification to the manipulator control regarding the current and/or upcoming change in the position of the mechanical elements of the tool. The manipulator control can thereby take into account the upcoming change in the position of these preferably powered mechanical elements before and during the next regulation step.

A mechanical element is preferably a linear axis. With a linear axis, extremely precise positionings and highly dynamic movements are possible. This is of particular relevance for applications in a medical manipulator, where work is performed on a very small area (inside the human body).

The aforementioned object is also solved by a method for the controlling of a medical manipulator comprising the following steps:

a. provision of a manipulator arm with an attached end effector comprising at least one tool with a varying mass and/or mass distribution;
b. controlling of the medical manipulator with the assistance of a manipulator control;
c. use of a current load data matrix of the varying mass and/or mass distribution of the tool in the manipulator control in each regulation step, in order to prevent deviations from a target posture or target path of the tool.

Through this method, the precision of the movements of the manipulator is increased and the ergonomics is improved for manually-controlled manipulator operations, in that the current values of the mass and mass distribution of the tool in each regulation step are taken into account in the manipulator control.

In the manipulator control, a dynamic model is preferably used, the calculation of which includes the varying mass and mass distribution of the tool. The manipulator control can thus determine the values for the mass distribution of the tool in each regulation step if it controls the mechanical elements itself. Additionally, values for future mass distributions can also be calculated according to the dynamic model and taken into account in the manipulator control.

The dynamic model of the tool is preferably integrated into the dynamic model of the manipulator. Through the use of a global dynamic model, fewer interfaces between the manipulator control and dynamic model(s) are required, which reduces the number of communication errors and the overall susceptibility of the control to error.

The manipulator control preferably takes into account the knowledge regarding a change in mass and mass distribution. The control method can thereby react more quickly to the error and correct it accordingly, namely already in the current and subsequent time step (regulator cycle). Additionally, the determination of the load data matrix can be optimized with respect to the data volume to be calculated, the number of calculations, or the speed, whereby shorter regulation times are possible.

The mass distribution is preferably calculated through the known position of one or more mechanical elements of the tool and then fed to the manipulator control. Because the position of the mechanical elements is generally very precisely determinable, the values of the mass distribution are also very precisely determinable. This leads to a very precise manipulator control overall.

A notification regarding a current and/or upcoming change in the position of the mechanical elements of the tool is preferably transmitted to the manipulator control by an electronic control unit that controls the mechanical elements. Because the electronic control unit already possesses the information regarding the upcoming change, on the basis of which it controls the mechanical elements accordingly in the next regulation step, it can also forward this information to the manipulator control, which for its part can take this information into account in advance in order to prevent the deviation from a target posture or target path of the tool.

### BRIEF DESCRIPTION OF THE DRAWINGS

The accompanying drawings, which are incorporated in and constitute a part of this specification, illustrate exemplary embodiments of the invention and, together with a general description of the invention given above, and the detailed description given below, serve to explain the principles of the invention.

FIG. 1 is a schematic construction of a medical manipulator according to the principles of the present disclosure.

### DETAILED DESCRIPTION

In the following, preferred embodiments of the present invention are described in detail with reference to the attached figure.

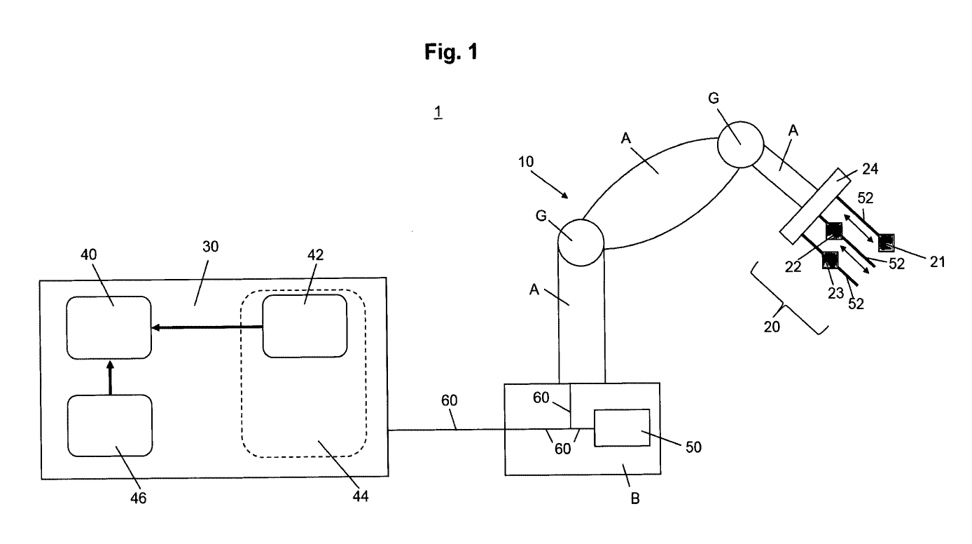

FIG. 1 shows an embodiment of a medical manipulator 1. A manipulator arm 10 is mounted on a base mounting surface B. In the illustrated embodiment, the manipulator arm 10 consists of three arm parts A and two joints G. However, the kinematic chain of the manipulator arm 10 can also comprise a different number of arm parts A and joints G, as desired. The manipulator arm 10 can be freely moved all spatial directions.

An end effector 20 comprising at least one tool 21, 22, 23 and a varying mass and/or mass distribution is mounted on the manipulator arm 10. Normally, the tools 21, 22, 23 are movable with respect to the manipulator arm 10 and upon such movements change their respective mass distribution relative to the manipulator arm 10. The tools 21, 22, 23 can, for example, be moved via mechanical elements 52 independently of the posture or movement of the manipulator arm 10. In the illustrated embodiment, the end effector 20 comprises three tools 21, 22, 23, which are moved individually via the mechanical elements 52, for example further into or out of the body of the patient. The mechanical elements 52 can be, for example, linear axes 52 with which the tools 21, 22, 23 can be individually operated in a linear fashion. However, the end effector 20 can also comprise a different number of tools 21, 22, 23, as desired. If the tools 21, 22, 23, in particular the medical instruments, are, for example, maximally inserted into the body of the patient, then the mass distribution of the tool is maximally towards the front. If all tools or instruments 21, 22, 23 are maximally extracted from the body of the patient, then the mass distribution is maximally towards the rear.

The mechanical elements 52 of the tools 21, 22, 23 are controlled by an electronic control unit 50. Via data links 60, the electronic control unit 50 preferably transmits information regarding the position of the mechanical elements 52 to the manipulator control 30.

The manipulator control 30 comprises a current load data matrix 40, which is preferably calculated by the control 30 from the current mass distributions as well as the masses of the tools 21, 22, 23. The current mass distributions as well as masses of the tools 21, 22, 23 can be stored as data 46 in the control 30. Based upon the use of the current load data matrix, the movement of the medical manipulator 1 is performed exactly according to the specifications of a user or a program. The current load data matrix 40 is also determined with the assistance of a dynamic model 42 of the tool(s) 21, 22, 23. The data 46 regarding the mass distribution and mass of the tool 21, 22, 23 can be fed from the medical manipulator 1 via the data link 60 in each regulation step to the manipulator control 30 and is calculated from the dynamic model 42 in each regulation step. The dynamic model of the tool 42 can also be integrated in the dynamic model of the manipulator 44. In particular, future changes in the mass distribution and the mass of the tool can be taken into account during the control of the manipulator 1.

In a method for the control of a medical manipulator 1, the load data matrix 40 is updated in each regulation step. By taking into account the current mass distribution and the mass 46, the manipulator control 30 can determine the forces and torques appearing on the tool 21, 22, 23 and correct the posture or path of the tool such that a deviation from an original target posture or target path is prevented.

In particular, the dynamic model 42, 44 can be extrapolated into the future in order to determine future values for the mass distribution 46. However, information regarding planned changes in the mechanical elements 52 can also be transmitted from the electronic control unit 50 to the manipulator control unit 30. This information can then be directly taken into account in the subsequent time step in the manipulator control.

As a result, the current control commands that take into account the change in the mass and mass distribution of the tool 21, 22, 23 are then transmitted in each regulation step via the data link 60 to the medical manipulator 1. A movement or posture of the tool 21, 22, 23 without a deviation from the target path or target posture can hereby be enabled, although the mass or mass distribution of the tool 21, 22, 23 varies.

While the present invention has been illustrated by a description of various embodiments, and while these embodiments have been described in considerable detail, it is not intended to restrict or in any way limit the scope of the appended claims to such detail. The various features shown and described herein may be used alone or in any combination. Additional advantages and modifications will readily appear to those skilled in the art. The invention in its broader aspects is therefore not limited to the specific details, representative apparatus and method, and illustrative example shown and described. Accordingly, departures may be made from such details without departing from the spirit and scope of the general inventive concept.

### LIST OF REFERENCE NUMBERS

1 Medical manipulator
10 Manipulator arm
20 End effector
21 Tool
22 Tool
23 Tool
24 Mounting surface
30 Manipulator control
40 Load data matrix
42 Dynamic model of the tool
44 Dynamic model of the manipulator
46 Mass distribution and mass of the tool
50 Electronic control unit
52 Mechanical elements
60 Data links
A Arm part
B Base mounting surface
G Joint

What is claimed is:

1. Medical manipulator (1), comprising:
a. a manipulator arm (10);
b. an end effector (20) mounted on the manipulator arm (10) and comprising at least one tool (21, 22, 23) with a varying mass and/or mass distribution (46);
c. a manipulator control (30) for the controlling of the medical manipulator (1); wherein
d. the manipulator control (30) uses a current load data matrix (40) with the varying mass and/or mass distribution (46) of the tool (21, 22, 23) in each regulation step in order to prevent deviations from a target posture or target path of the tool (21, 22, 23).

2-14. (canceled)
